(19) United States
(12) Patent Application Publication
(10) Pub. No.: US 2010/0099757 A1
(43) Pub. Date: Apr. 22, 2010

(54) DISULFIRAM DOSES AND TREATMENT REGIMEN SUITABLE FOR TREATMENT OF ANGIOGENESIS-DEPENDENT DISORDERS

(76) Inventors: Yossef Israeli-Shalev, Chakuk (IL); Amichai Bar-On, Tel Aviv (IL)

Correspondence Address: FENNEMORE CRAIG, 3003 NORTH CENTRAL AVENUE, SUITE 2600, PHOENIX, AZ, 85012, US

(21) Appl. No.: 12/517,266

(22) PCT Filed: Dec. 2, 2007
(86) PCT No.: PCT/IL07/01487

§ 371 Date: Sep. 15, 2009

Related U.S. Application Data
(60) Provisional application No. 60/872,513, filed on Dec. 4, 2006.

(51) Int. Cl. A61K 31/27; C07C 333/20

(52) U.S. Cl. 514/483; 564/76

(57) ABSTRACT

A pharmaceutical composition is provided. The pharmaceutical composition includes an amount of disulfuram in unit dosage form which results in a peak serum concentration level which is therapeutically effective in treating an angiogenesis-dependent disorder and can also exhibit minimal or no disulfuram related side effects.

### FIELD AND BACKGROUND OF THE INVENTION

The present invention relates to therapeutic methods utilizing disulfuram for the treatment of cancer and other angiogenesis-dependent disorders in humans, specifically to doses and schedules suitable for treatment of cancer.

Angiogenesis, the growth of new capillary blood vessels by sprouting from established vessels, requires the growth of vascular endothelial cells and vascular smooth muscle cells. Tumor development is known to largely depend on angiogenesis. As a result, inhibition of angiogenesis was and is a target for treatment of cancer. Bis(diethylthiocarbamoyl) disulfide or tetraethylthiuram disulfide, hereinafter disulfuram, is a chelator of heavy metals and the active ingredient in the drug Antabuse® used for many years in aversion therapy for chronic alcoholism. Disulfuram inhibits activity of Cu/Zn Suproxide dismutase (SOD-1) both in vivo and in vitro (Marikovsky et al., 2002). Oral administration of disulfuram inhibited angiogenesis in CD1 nude mice (Marikovsky et al., 2002). Application of disulfuram to cultured bovine capillary endothelial (BCE) cells caused inhibition of DNA synthesis and induction of apoptosis. These effects were prevented by addition of copper and antioxidants indicating involvement of reactive oxygen species (ROS) and SOD-1. Disulfuram also reduced the level of glutathione in BCE cells. Oral administration of disulfuram to mice caused significant inhibition of C6 glioma tumor development and marked reduction (by 10-19 fold) in the metastatic load of Lewis lung carcinoma. The data establishes disulfuram as a potential inhibitor of angiogenesis and of angiogenesis-dependent pathologies such as solid tumors. Independent scientific groups received similar results with disulfuram as a potential inhibitor of cancer (see, for Example, Shine-Gwo et al., 2003; Zuben et al., 2004; Loo et al., 2000; Dazhi et al., 2002; Weiguang et al., 2003; Carina et al., 2003; Cen et al., 2004).

Disulfuram was approved by the U.S. Food and Drug administration (Antabuse®) to be used as an aid in the management of selected chronic alcoholic patients who want to remain in a state of enforced sobriety so that supportive and psychotherapeutic treatment may be applied to best advantage. The therapeutic use of disulfuram for alcoholism was discovered in the 1930s when workers exposed to disulfuram, a chemical used in the rubber industry, became ill after drinking alcoholic beverages. Disulfuram produces sensitivity to alcohol, which results in a highly unpleasant reaction when the patient under treatment ingests even small amounts of alcohol.

In the treatment of alcohol addiction a dose of 250-500 mg/day is given for prolonged periods of time. Antabuse can be purchased in the US in 250 mg tablets for oral administration from Odyssey Pharmaceuticals, Inc., East Hanover, N.J. 07936.

U.S. Pat. No. 6,288,110 to Marikovsky discloses therapeutic methods utilizing disulfuram in the treatment of angiogenesis-dependent disorders, including neoplasms, and for preventing cell hyperproliferation and formation of clots along or around medical devices. The '110 patent demonstrates, inter alia, inhibition of Lewis Lung Carcinoma and C6 Glioma Tumor Growth in mice.

U.S. Pat. No. 6,589,987 to Kennedy is directed to a method for using tetraethylthiuram disulfide to reduce tumor growth, and to potentiate the effect of other anticancer agents. Specifically, the '987 patent discloses a method for treating an established cancer sensitive to the enhanced combination of tetraalkyl thiuram disulfide and a heavy metal ion comprising administering said combination to a mammal in a therapeutically effective amount and wherein the heavy metal ion is a copper ion. The specification of '987 discloses that a combination of disulfuram and metal ions inhibited the proliferation of human malignant cell lines in vitro. Based on a correlation between the effective concentration of disulfuram that inhibited cell proliferation in vitro and the serum levels of disulfuram previously reported upon administration of 250 mg disulfuram to alcoholics (Faiman et al., 1984), a preferred unit dose from about 250 mg to about 500 mg, was suggested.

Several attempts to examine disulfuram clinically for the treatment of cancer in humans were reported.

Barr et al. (2004) discloses a case report wherein disulfuram and Zn+2 were used on a subject having advanced stage IV metastatic melanoma. The subject received a daily dose of 250-500 mg disulfuram and 50 mg zinc gluconate. A >50% reduction in tumor size was reported after three months from the onset of therapy.

As preclinical studies have indicated that disulfuram may be useful for decreasing the toxicity and enhancing the therapeutic index of the chemotherapeutic drug cisplatin (Borch et al., 1979; Borch et al., 1981; Borch et al., 1984), Phase I (Stewart et al., 1987) and Phase II (Verma et al., 1990) clinical trials of disulfuram and cisplatin as a treatment for cancer were performed. A dose of about 2000 mg/subject disulfuram combined with 100 mg/m2 cisplatin, as determined from the dose-limiting toxicity, was administered to the subjects once every 3-4 weeks. However, no statistically-significant difference was found in response rate, time to progression, or median survival between the group receiving cisplatin in combination with disulfuram and the group receiving cisplatin alone. Moreover, disulfuram did not afford significant nephroprotection against cisplatin and, in fact, enhanced gastrointestinal and ototoxicities.

While the prior art offered evidence of the potential usefulness of disulfuram in the treatment of neoplastic disorders, it fails to demonstrate safe and effective use of disulfuram for the treatment of cancer in humans at low doses. In addition, the prior art does not teach or suggest treatment doses that are substantially lower than those utilized for alcohol abuse treatment.

While reducing the present invention to practice, the present inventors have uncovered that doses of disulfuram which are substantially lower than those described in the prior art are effective in treating disorders caused by aberrant angiogenesis.

### SUMMARY OF THE INVENTION

According to one aspect of the present invention there is provided a pharmaceutical composition comprising an amount of disulfuram in unit dosage form which results in a peak serum concentration level of disulfuram or a metabolite thereof which is less than 0.5 μM.

According to another aspect of the present invention there is provided a pharmaceutical composition comprising a unit dose form including about 10-200 mg of disulfuram.

According to further features in preferred embodiments of the invention described below, the pharmaceutical composition comprises about 30-120 mg of the disulfuram.

According to still further features in the described preferred embodiments the unit dose form is formulated for oral administration.

According to still further features in the described preferred embodiments the pharmaceutical composition further comprises a pharmaceutical carrier.

According to still further features in the described preferred embodiments the pharmaceutical carrier is devoid of alcohol and/or metal ions.

According to another aspect of the present invention there is provided a pharmaceutical composition comprising an amount of disulfuram in unit dosage form which does not substantially cause a disulfuram-related side effect in a subject.

According to still further features in the described preferred embodiments the disulfuram-related side effect is alcohol toxicity.

According to still further features in the described preferred embodiments the amount is effective in inhibiting angiogenesis.

According to still further features in the described preferred embodiments the amount is effective in inhibiting metastasis.

According to still further features in the described preferred embodiments the amount is effective in inhibiting tumor development.

According to another aspect of the present invention there is provided a method of treating an angiogenesis-dependent disorder, comprising administering to a subject in need a unit dose of disulfuram which does not substantially cause a disulfuram-related side effect in the subject at least once every three days thereby treating the angiogenesis-dependent disorder.

According to still further features in the described preferred embodiments the disulfuram is administered daily.

According to still further features in the described preferred embodiments the unit dose of disulfuram is about 10 mg to about 200 mg. other ranges, such as for example, 10-180, 20-160, 60-120 or 60-100 mg are also contemplated herein.

According to still further features in the described preferred embodiments the subject in need is predisposed to or suffering from cancer, an opthalmologic disorder, a dermatologic disorder, a pediatric disorder, an orthopedic disorder, a neurologic cerebrovascular disorder, a connective tissue disorder or a hypertrophic scar.

According to still further features in the described preferred embodiments the cancer is non-small cell lung cancer.

According to still further features in the described preferred embodiments the disulfuram is administered to the subject in need thereof in combination with at least one other anti-cancer agent.

According to still further features in the described preferred embodiments the anti-cancer agent is selected from the group consisting of alkylators, anthracyclines, antibiotics, aromatase inhibitors, bisphosphonates, cyclo-oxygenase inhibitors, estrogen receptor modulators, folate antagonists, inorganic aresenates, microtubule inhibitors, nitrosoureas, nucleoside analogs, osteoclast inhibitors, platinum containing compounds, retinoids, topoisomerase 1 inhibitors, topoisomerase 2 inhibitors, and tyrosine kinase inhibitors.

According to still further features in the described preferred embodiments the chemotherapeutic agent is selected from the group consisting of platinum-containing chemotherapy and taxanes.

According to another aspect of the present invention there is provided an article of manufacturing comprising a unit dose form including about 10-180 mg of disulfuram and packaging material identifying the unit dose for use in treating an angiogenesis-dependent disorder.

According to still further features in the described preferred embodiments the unit dose includes about 30-120 mg of disulfuram.

According to still further features in the described preferred embodiments the packaging material identifies the unit dose for use in treating non-small cell lung cancer.

According to still further features in the described preferred embodiments the subject is not suffering from alcohol abuse.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### DESCRIPTION OF THE PREFERRED EMBODIMENTS

The present invention is of a disulfuram doses and treatment regimen which can be used to treat disorders such as cancer. Specifically, the present invention can be used to treat cancer using doses of disulfuram substantially lower than those utilized in the prior art.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Disulfuram has been used for treating alcohol abuse for more than 50 years, as Antabuse™ which was introduced in 1948. The human therapeutic dose of disulfuram ranges from 125 to 500 mg/day. When combined with alcohol consumption, Disulfuram produces an aversive side effect, typically at a dose ranging between 250 mg/day and 500 mg/day.

Although prior art studies provide evidence of the potential usefulness of disulfuram in the treatment of cancer, such studies typically suggest that disulfuram can be effective when administered at an amount within the conventional clinical ranges prescribed for alcohol abuse treatment and thus do not teach or suggest use of disulfuram at substantially lower doses.

Disulfuram, or bis(diethylthiocarbamoyl) disulfide, has the following chemical: formula:

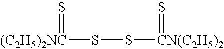

Disulfuram occurs as a white to off-white, odorless, and almost tasteless powder which is soluble in water to the extent of about 20 mg in 100 ml, and in alcohol to the extent of about 3.8 g in 100 ml. Disulfuram is marketed as 200, 250, 500 mg dose units (tablets) under the trade name Antabuse™.

Disulfuram blocks the conversion of alcohol to acetic acid at the intermediate stage by blocking the enzyme acetaldehyde dehydrogenase which converts acetaldehyde, a primary breakdown product of alcohol, to acetic acid. After alcohol intake under the influence of disulfuram, the concentration of acetaldehyde in the blood may be 5 to 10 times higher than that found during metabolism of the same amount of alcohol alone. As acetaldehyde is one of the major causes of the symptoms of a “hangover” this produces immediate and severe negative reaction to alcohol intake. Some 5-10 minutes after alcohol intake, a patient may experience the effects of a severe hangover for a period of 30 minutes up to several hours. Symptoms include flushing of the skin, accelerated heart rate, shortness of breath, nausea, and vomiting.

In order to cause such side effects, the desirable peak plasma concentration of disulfuram should typically be within a range of about 0.5 to about 5

The onset of effect for a single dose unit is within 1 to 2 hours. The peak effect occurs at about 12 hours and is sustained for 12-72 hours depending on the activity and concentration of the liver enzymes. In some individuals, the effects of a single dose can last up to 2 weeks (Huffman J C, Stern T A. Disulfuram Use in an Elderly Man with alcoholism and heart disease: A Discussion. Primary Care Companion J Clin Psychiatry 2003; 5:41-4).

In the body, disulfuram is converted into diethylthiomethylcarbamate (Me-DTC) through 3 intermediate steps. Me-DTC is considered to be most powerful irreversible inhibitor of aldehyde dehydrogenase (Hart B W, Yourick J J, Faiman M D. S-methyl-N,N-diethylthiolcarbamate: A disulfuram metabolite and potent rat liver mitochondrial low Km aldehyde dehydrogenase inhibitor. Alcohol 1990; 7:165-9).

Usually the symptoms of disulfuram ethanol reaction (DER) last for 30-120 min, depending upon the dose and amount of ethanol consumed (Petersen E N. The pharmacology and toxicology of disulfuram and its metabolite. Acta Psychiatr Scand Suppl 1992; 369:7-13) (Sauter A M, Boss D, von Wartburg J P. Reevaluation of the disulfuram-alcohol reaction in man. J Stud Alcohol 1977; 38:1680-95). The threshold for the reaction is approximately 1 ml of 100% ethanol or its equivalent (Eneanya D I, Bianchine J R, Duran D O, Andresen B D. The actions and metabolic fate of disulfuram. Ann Rev Pharmacol Toxicol 1981; 21:575-96).

While studying the effect of disulfuram on cancer patients, the present inventors have unexpectedly discovered that doses of disulfuram substantially lower than prescribed for alcohol abuse or suggested in the art, are clinically effective for the treatment of cancer.

Specifically, experiments conducted by the present inventors have shown that administration of 120 mg disulfuram daily (unit dose of 40 mg three times a day) for a period of eighteen weeks can be used for extending time to progression and survival of non-small cell lung cancer in human patients.

Thus, the present invention provides novel disulfuram dose units and methods of treating angiogenesis-dependent disorders using a treatment regimen which employ such dosing.

As used herein, the phrase “angiogenesis-dependent disorder” refers to a disorder in which inhibition of angiogenesis is advantageous in treating or reversing the disease state or condition. The angiogenesis-dependent disorder includes, but is not limited to, an opthalmologic disorder (e.g. diabetic retinopathy, age related macular degeneration, corneal graft neovascularization, neovascular glaucoma, trachoma and retinopathy of prematurity also known as retrolental fibroplasias), a dermatologic disorder (e.g. dermatitis and pyogenic granuloma), a pediatric disorder (e.g. hemangioma, angiofibroma, and hemophilic joints), an orthopedic disorder (e.g. nonunion fractures), a neurologic cerebrovascular disorder (e.g. arteriovenous malformation), or a connective tissue disorder (e.g. scleroderma, and hypertrophic scars).

The angiogenesis-dependent disorder can also be a cancer, including solid tumors and blood borne neoplasms. Examples includes, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung cancer, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, and the metastatic lesions secondary to these primary tumors, and benign solid tumors such as acoustic neuroma, neurofibroma, trachoma and pyogenic granulomas.

Dose units of the disulfuram composition of the present invention are selected so as to provide a therapeutic effect and preferably also minimize any disulfuram related side effect, such as nausea.

Preferred dose units can include 10-200 or 60-10 mg of disulfuram and can be administered once to several times daily. Preferably, dose units and dosing is selected so as to result in peak serum concentration level of less than 0.5 μM disulfuram more preferably between 0.05-0.5 μM. Faiman et al. (Clinical Pharmacol. Ther. Oct. 1984) reported that there is a marked inter-subject variability in the plasma concentration of disulfuram and its metabolites after disulfuram intake. Elimination kinetics of oral DSF demonstrated that T1/2 is at 7.3 hours and the average time to reach maximum plasma concentration was 8-10 hours. In order to maximize an anti-angiogenic effect, it is best to obtain a steady serum level of disulfuram.

Thus, the present inventors postulate that in order to provide for subject variability and maximize an anti-angiogenic effect, typical dose units amounts should be between 1-200 mg, preferably, 5-180 mg, more preferably 10-140 mg, most preferably 30-120 mg. Other ranges such as 50-180 mg or 60-120 mg are also preferred.

Depending on the disorder and its severity and the subject treated, such dose units can be administered once-daily or more/less than once daily. Preferably, a unit dose of 40 or 60 mg is administered three times daily for a total daily dose of 120 or 180 mg.

In any case, an individual is treated such that over the treatment period the peak serum concentration of disulfuram does not exceed 0.5 μM over the treatment period. Detailed descriptions of treatment regimen using the disulfuram dose units of the present invention are provided below and in the Examples section which follows.

The dose units of the present invention are formulated as pharmaceutical compositions which include disulfuram and a pharmaceutically acceptable carrier. The term “pharmaceutically acceptable carrier” refers to a vehicle which delivers the active components to the intended target and which does not cause harm to humans or other recipient organisms. It should be noted, that in certain cases, the use of alcohols as carriers in disulfuram-containing compositions may cause undesired or adverse effects to the subject in need of treatment. Thus, pharmaceutical compositions utilizing non-alcoholic carriers are preferred. In addition, since disulfuram is a metal chelator, preferred carriers are those devoid of zinc or copper ions.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration are in dosages suitable for the chosen route of administration.

The unit dose forms of the present invention may, if desired, be presented in a pack or dispenser device, such as an FDA approved kit, which may contain one or more unit dose forms containing the active ingredient. The pack may, for example, comprise metal or plastic foil, such as a blister pack. The pack or dispenser device may be accompanied by instructions for administration, and treatment regimen. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of the indicated disorder as is detailed hereinunder.

The present invention further provides methods of treating and ameliorating angiogenesis dependent disorders, as detailed herein. One presently preferred treatment target is lung cancer, such as, non-small-cell lung cancer (NSCLC). NSCLC is currently treated by radiation therapy, surgery, and/or chemotherapy. Simultaneous administration of disulfuram as described herein can improve treatment outcomes for all current therapies.

As used herein, “treating” an angiogenesis-dependent disorder, e.g. cancer (or treating a subject with or predisposed to, the disorder) refers to taking steps to obtain beneficial or desired results, including but not limited to, alleviation or amelioration of one or more symptoms of the disorder, diminishment of extent of disorder, prevention of the onset of the disorder, delay or slowing of progression, amelioration, palliation or stabilization of the disorder, partial or complete remission, prolonged survival and other beneficial results known in the art.

According to one embodiment of the invention, treatment is effected by administering to a subject in need of treatment for a cancer a therapeutically effective dose of disulfuram, where the therapeutically effective dose is obtained by administering disulfuram at a frequency greater than once every two or three days.

Preferably, disulfuram is administered at a daily dose of about 1 mg of to about 200 mg for multiple days. It should be noted that a “daily dose” can be achieved via one or more daily administrations. Thus, a daily dose of disulfuram can be divided into subdoses and administered in multiple doses, e.g., twice (bid), three times (tid), or four times (qid) per day; for example, a daily dose of 180 mg can be achieved by administration of 3 dose units, 60 mg each.

According to preferred embodiments of the present invention, disulfuram is administered in a daily dose of about 60 mg to about 120 mg per subject.

Disulfuram can be administered for a period of days, weeks or months using continual or intermittent treatment regimes. Continual treatment refers to treatment on a daily basis without interruption, while intermittent treatment refers to treatment that is not continuous, but rather cyclic in nature.

A subject may be administered disulfuram in accordance with the present methods for several days, a week, a month, two months, four months, six months, or a year or longer.

Disulfuram is preferably administered in the absence of alcohol or zinc or copper ions. The effective and safe use of disulfuram for the treatment of cancer in humans in the absence of such ions was demonstrated for the first time by the present inventors.

Disulfuram can be administered alone or in combination with at least one other anti-cancer agent. The anti-cancer agent can be a chemotherapeutic drug, such as, alkylators including, but not limited to, busulfan (Myleran, Busulfex), chlorambucil (Leukeran), ifosfamide (with or without MESNA), cyclophosphamide (Cytoxan, Neosar), glufosfamide, melphalan, L-PAM (Alkeran), dacarbazine (DTIC-Dome), and temozolamide (Temodar); anthracyclines, including, but not limited to doxorubicin (Adriamycin, Doxil, Rubex), mitoxantrone (Novantrone), idarubicin (Idamycin), valrubicin (Valstar), and epirubicin (Ellence); antibiotics, including, but not limited to, dactinomycin, actinomycin D (Cosmegen), bleomycin (Blenoxane), daunorubicin, and daunomycin (Cerubidine, DanuoXome); aromatase inhibitors, including, but not limited to anastrozole (Arimidex) and letroazole (Femara); bisphosphonates, including, but not limited to zoledronate (Zometa); cyclo-oxygenase inhibitors, including, but not limited to, celecoxib (Celebrex); estrogen receptor modulators including, but not limited to tamoxifen (Nolvadex) and fulvestrant (Faslodex); folate antagonists including, but not limited to methotrexate and tremetrexate; inorganic aresenatesincluding, but not limited to arsenic trioxide (Trisenox); microtubule inhibitors (e.g. taxanes) including, but not limited to vincristine (Oncovin), vinblastine (Velban), paclitaxel (Taxol, Paxene), vinorelbine (Navelbine), epothilone B or D or a derivative of either, and discodermolide or its derivatives, nitrosoureas including, but not limited to procarbazine (Matulane), lomustine, CCNU (CeeBU), carmustine (BCNU, BiCNU, Gliadel Wafer), and estramustine (Emcyt); nucleoside analogs including, but not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda); osteoclast inhibitors including, but not limited to pamidronate (Aredia); platinum containing compounds including, but not limited to cisplatin (Platinol) and carboplatin (Paraplatin); retinoids including, but not limited to tretinoin, ATRA (Vesanoid), alitretinoin (Panretin), and bexarotene (Targretin); topoisomerase 1 inhibitors including, but not limited to topotecan (Hycamtin) and irinotecan (Camptostar); topoisomerase 2 inhibitors including, but not limited to etoposide, VP-16 (Vepesid), teniposide, VM-26 (Vumon), and etoposide phosphate (Etopophos); and tyrosine kinase inhibitors including, but not limited to imatinib (Gleevec).

Preferred anti-cancer agents are platinum-containing chemotherapeutics and taxanes. In addition, such drugs are preferably provided in carriers which are devoid of alcohol for the reasons stated hereinabove.

Co-administration of disulfuram and the anti-cancer agent can be effected by concurrent or stepwise administration of disulfuram and the anti-cancer agent.

Thus, for example, disulfuram and the anti-cancer agent can be administrated as a single co-formulation or disulfuram may be administered prior to, or following administration of the anti-cancer drug. In the latter case, disulfuram treatment can be maintained throughout the course of the anti-cancer agent treatment or during a part thereof. In certain preferable embodiments (in high risk patients or patients having recurrence after primary cancer therapy), disulfuram treatment may be continued following cessation of the anti-cancer agent treatment or it may be initiated and completed prior to or following the administration of the anti-cancer agent.

Disulfuram can be administered to the subject in need in combination with at least two other anti-cancer agents. In such cases, the anti-cancer agents are preferably platinum-containing chemotherapeutics and taxanes.

For example, current treatment regimens for non-small-cell lung cancer include without limitation administration of Gemcitabine (Eli Lilly, difluorodeoxy-cytidine), vinorelbine, paclitaxel, docetaxel, cisplatin, carboplatin, or Irinotecan (camptothecin-11) as single agents; and administration of etoposide and cisplatin, Vindesine (deacetyl vinblastine carboxamide) and cisplatin, paclitaxel and carboplatin, Gemcitabine and carboplatin, docetaxel and cisplatin, vinorelbine and cisplatin, or Irinotecan and cisplatin in combination therapies (see Bunn 2002, incorporated herein by reference). In accordance with the methods of the present invention, disulfuram can be co-administered in such therapeutic regimens to improve subject outcomes. For example and without limitation, co-administration of disulfuram as described herein can improve the following chemotherapy for NSCLC: administration of cisplatin at 80 mg/m2, IV for on day one and once every three weeks and vinorelbine at 25 mg/m2, IV on days 1, 8 and once every three weeks for six cycles, wherein disulfuram treatment may optionally continue beyond the administration period of cisplatin and vinorelbine.

A detailed description of a treatment regimen which follows the teachings of the present invention is provided in the Examples section below.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Clinical Trial Protocol for Lung Cancer Therapy

Trial Design

A phase II, multicenter, randomized, double blind study was designed to assess the dose response of humans suffering from lung cancer to Disulfuram or a disulfuram-Cisplatin/Vinorelbine combination. Patients with histogically or cytologically documented advanced (i.e. stage Mb or stage IV), or recurrent non-small cell lung cancer, previously untreated with systemic anti-tumor therapy were selected for the study. The patients were randomly assigned to the standard first line hospital chemotherapy treatment (Cisplatin/Vinorelbine) with placebo or Cisplatin/Vinorelbine in combination with disulfuram. Treatment was provided in cycles of 21-day time periods until progression or for a maximum of 6 cycles.

TABLE 1
treatment program of the trial

| | Drug Treatment | |
|---|---|---|
| | Treatment continues in the absence of disease progression, | |
| Screening | unacceptable toxicity or patient refusal | Follow- up |
| Baseline | Cisplatin, 80 mg/m², IV on day 1 and once every 3 weeks | Patients are |
| studies are | Vinorelbine, 25 mg/m², IV on days 1, 8 and once every 3 weeks | followed every |
| performed | Disulfiram, 40 mg × 3 a day, PO on day 1 and every day | 12 weeks |
| within 28 days | | |
| before patient | | |
| enrolment | | |

Exclusion Criteria:

Patients were excluded from the trial for the following reasons:

Current consumption of alcohol due to known ethanol abuse or use of alcohol-containing medication.

Patients who are receiving or have recently received (for at least 12 hours) metronidazole, paraldehyde, alcohol, or alcohol-containing preparations, e.g., cough syrups, tonics and the like.

Any known co-morbid psychiatric disorder that met DSM V criteria excluding nicotine dependence;

Known hypersensitivity to any of the study drugs;

Prior chemotherapy or prior treatment with systemic anti-cancer agent (e.g. monoclonal antibody, tyrosin kinase inhibitor). Note: prior surgery and irradiation are permitted;

Pregnancy or lactation in women;

Fertile men or women of childbearing potential not using adequate contraception;

Malignancies other than NSCLC within 5 years prior to randomization, except for adequately treated carcinoma in situ of the cervix; basal or squamous cell skin cancer; localized prostate cancer treated surgically with curative intent; DCIS treated surgically with curative intent;

Treatment with any other investigational agent, or participation in any other clinical trial within 30 days prior to entering this study;

Evidence of any other disease, metabolic dysfunction, physical examination finding or laboratory finding giving reasonable suspicion of a disease or condition that contra-indicates the use of the investigational drug or puts the patients in high risk for treatment related complications;

Evidence for liver dysfunction—bilirubin level twice the normal value;

Patients with a history of rubber contact dermatitis;

Patients receiving phenyloin and its congeners;

Patients taking isoniazid;

Brain metastases or spinal cord compression.

Table 2 below summarizes the procedures and assessments conducted throughout the trial.

TABLE 2
schedule of procedures and assessments performed throughout the trial

| Test | Baseline within 4 weeks before therapy | Day 1 | Day 8 | Every 3 weeks | Every 12 weeks |
|---|---|---|---|---|---|
| Informed consent | ✓ | | | | |
| Medical history | ✓ | | | | |
| Physical examination | ✓ | ✓ | ✓ | ✓ | |
| 12-lead ECG | ✓ | | | | |
| Tumor assessment by CT scan | ✓ | | | | ✓ |
| ECOG PS (XXXX) | ✓ | ✓ | ✓ | ✓ | |
| QOL (quality of life) | ✓ | | | ✓ | |
| Serum pregnancy test (female only) | ✓ (7 days before first treatment) | | | | |
| Hematology | ✓ (7 days before first treatment) | ✓ | ✓ | ✓ | |
| Biochemistry | ✓ (7 days before first treatment) | | ✓ | ✓ | |
| Urinalysis | ✓ (7 days before first treatment) | | | ✓ | |
| Adverse events | | ✓ | ✓ | ✓ | ✓ |
| Serum test for prothrombotic & | ✓ | ✓ | ✓ | ✓ | |
| endothelial cell activation | | | | | |
| markers (angiogenesis markers) | | | | | |

The patients were further assessed for overall survival, time to progression and duration of response.

Safety

Serious side effects (clinically) or abnormal laboratory tests were recorded on a case report form (intensity, relationship to disulfuram, action taken, and outcome to date); patients showing any severe side effects from disulfuram are withdrawn from the therapy. In addition, patients showing decrease in the Karnofsky score (Hollen et al. Cancer. 1994; 73: 2087-2098) by 20 points or more are considered to have progressed on disulfuram and are withdrawn from the therapy.

Standard dosing adjustment was followed according to laboratory tests/toxicity grades.

Standard administration regimens for cisplatin and Vinorelbine were followed. Disulfuram/placebo was administered at doses of 40 mg×3/day p.o. daily throughout the duration of the study, until progression. Treatment with disulfuram is continued until patient's withdrawal due to disease progression or toxicity or patient refusal to continue therapy.

Disulfuram (tetraethylthiuram disulfide, 25 kg pharmaceutical grade) was purchased through Tamar Marketing under the Israeli Ministry of Health regulations. Capsules (disulfuram & placebo) were prepared locally by mixing 40 mg of disulfuram with a lactose carrier. Placebo capsules included the lactose carrier but were devoid of disulfuram. Each patient received the disulfuram or placebo from the treating physician.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Marikovsky M, Nevo N, Vadai E, Harris-Cerruti C. CU/ZN superoxide dismutase plays a role in angiogenesis. Int. J. Cancer 2002; 97: 34-41
2. Shine-Gwo S, Yu-Rung K, Felicia W U, Cheng-Wen W U. Inhibition of Invasion and Angiogenesis by Zinc-Chelating Agent Disulfuram. Mol Pharmacol 2003; 64:1076-1084
3. Zuben E S, Xiang-Hong P, Krishnamachary N, Samrawit T, Suresh V A. The Molecular Basis of the Action of Disulfuram as a Modulator of the Multidrug Resistance-Linked ATP Binding Cassette Transporters MDR1 (ABCB1) and MRP1 (ABCC1). Mol Pharmacol: 2004; 65:675-684
4. Loo W L, Clarke D M. Blockage of drug resistance in vitro by disulfuram, a drug used to treat alcoholism. J. National Cancer Inst 2000; 92 (11): 898
5. Dazhi C, Rachel I G, Julie A B, Ravi S K, Nilou B T, Frank L M, Jr. Disulfuram Induces Apoptosis in Human Melanoma Cells: A Redox-related Process. Mol Cancer Ther 2002; 1: 197-204
6. Weiguang W, Howard L. M, James C. Disulfuram-Mediated Inhibition of NF-KB Activity Enhances Cytotoxicity of 5-Fluorouracil in Human Colorectal Cancer Cell Lines. Int. J. Cancer 2003; 104, 504-511
7. Carina T, John P. F, Lan H, Ricardo P. Enhanced Antimelanoma Activity After Exposure to BSO in Combination with Disulfuram. American Association for Cancer Research (AACR) 2003
8. Cen D, Brayton D, Shahandeh B, Meyskens F L Jr, Farmer P J. Disulfuram facilitates intracellular Cu uptake and induces apoptosis in human melanoma cells. J Med. Chem. 2004; 47(27): 6914-20
9. Brar S S, Grigg C, Wilson K S, Holder W D Jr, Dreau D, Austin C, Foster M, Ghio A J, Whorton A R, Stowell G W, Whittall L B, Whittle R R, White D P, Kennedy T P. Disulfuram inhibits activating transcription factor/cyclic AMP-responsive element binding protein and human melanoma growth in a metal-dependent manner in vitro, in mice and in a patient with metastatic disease. Mol Cancer Ther. 2004; 3(9): 1049-60
10. Borch R F, Katz J C. Effect of diethyldithiocarbamate (DDTC) or disulfuram (DSF) rescue on the response of P388 leukemia to cis-dichlorodiammine platinum (II) (DDP) in BDF mice. Proc Am Assoc Cancer Res 1981; 22:259
11. Borch R F, Pleasents M E. Inhibition of cis-platinum nephrotoxicity by diethyldithiocarbamate rescue in rat model. Proc Natl Acad Sci 1979; 76:661-4
12. Borch R F, Bodenner D L, Katz J C. Diethyldithiocarbamate and cisplatin toxicity. In: Hacker M P, Douple E B, Krakoff I H, eds. Platinum coordination complexes in cancer chemotherapy. Boston: Maritnus-Nijhoff; 1984:154-64
13. Verma S, Stewart D J, Maroun J A, Nair R C. A randomized phase II study of cisplatin alone versus cisplatin plus disuliram. Am J Clin Oncol 1990; 13(2): 119-124
14. Stewart D J, Verma S, Maroun J A. Phase I study of the combination of disulfuram with cisplatin. Am J Clin Oncol 1987; 10(6): 517-9
15. Bunn, J. Clin. One. 2002; 20(18s): 23-33

What is claimed is:

1. A pharmaceutical composition comprising an amount of disulfuram in unit dosage form which results in a peak serum concentration level of disulfuram or a metabolite thereof which is less than 0.5 μM.

2. A pharmaceutical composition comprising a unit dose form including about 10-180 mg of disulfuram.

3. The pharmaceutical composition of claim 2, comprising about 30-120 mg of said disulfuram.

4. The pharmaceutical composition of claim 2, comprising 60-120 mg of said disulfuram.

5. The pharmaceutical composition of claim 2, wherein said unit dose form is formulated for oral administration.

6. The pharmaceutical composition of claim 2, further comprising a pharmaceutical carrier.

7. The pharmaceutical composition of claim 6, wherein said pharmaceutical carrier is devoid of alcohol and/or metal ions.

8. A pharmaceutical composition comprising an amount of disulfuram in unit dosage form which does not substantially cause a disulfuram-related side effect in a subject.

9. The pharmaceutical composition of claim 8, wherein said disulfuram-related side effect is alcohol toxicity.

10. The pharmaceutical composition of claim 8, wherein said amount is effective in inhibiting angiogenesis.

11. The pharmaceutical composition of claim 8, wherein said amount is effective in inhibiting metastasis.

12. The pharmaceutical composition of claim 8, wherein said amount is effective in inhibiting tumor development.

13. A method of treating an angiogenesis-dependent disorder, comprising administering to a subject in need a unit dose of disulfuram which does not substantially cause a disulfuram-related side effect in said subject at least once every three days thereby treating the angiogenesis-dependent disorder.

14. The method of claim 13, wherein said disulfuram is administered daily.

15. The method of claim 13, wherein said unit dose of disulfuram is about 10 mg to about 180 mg.

16. The method of claim 13, wherein said unit dose of disulfuram is about 30 mg to about 120 mg.

17. The method of claim 13, wherein said subject in need is predisposed to or suffering from cancer, an opthalmologic disorder, a dermatologic disorder, a pediatric disorder, an orthopedic disorder, a neurologic cerebrovascular disorder, a connective tissue disorder or a hypertrophic scar.

18. The method of claim 17, wherein the cancer is non-small cell lung cancer.

19. The method of claim 13, wherein said disulfuram is administered to said subject in need thereof in combination with at least one other anti-cancer agent.

20. The method of claim 19, wherein the anti-cancer agent is selected from the group consisting of alkylators, anthracyclines, antibiotics, aromatase inhibitors, bisphosphonates, cyclo-oxygenase inhibitors, estrogen receptor modulators, folate antagonists, inorganic aresenates, microtubule inhibitors, nitrosoureas, nucleoside analogs, osteoclast inhibitors, platinum containing compounds, retinoids, topoisomerase 1 inhibitors, topoisomerase 2 inhibitors, and tyrosine kinase inhibitors.

21. The method of claim 20, wherein the chemotherapeutic agent is selected from the group consisting of platinum-containing chemotherapy and taxanes.

22. The method of claim 13, wherein said subject is not suffering from alcohol abuse.

23. An article of manufacture comprising a unit dose form including about 10-180 mg of disulfuram and packaging material identifying said unit dose for use in treating an angiogenesis-dependent disorder.

24. The article of manufacture of claim 23, wherein said unit dose includes about 30-120 mg of disulfuram.

25. The article of manufacture of claim 23, wherein said packaging material identifies said unit dose for use in treating non-small cell lung cancer.
